(19) United States
(12) Patent Application Publication
(10) Pub. No.: US 2024/0181460 A1
(43) Pub. Date: Jun. 6, 2024

(54) HIGHLY SEALED PCR SINGLE TUBE

(71) Applicant: Meidong Huicheng Life Technogy (Kunshan) Co., Ltd., Suzhou, Jiangsu (CN)

(72) Inventors: GANG LIU, Suzhou, Jiangsu (CN); ZIXIAN LIU, Suzhou, Jiangsu (CN); ZIDE LIU, Suzhou, Jiangsu (CN); YIYONG YUAN, Suzhou, Jiangsu (CN); HUA ZHAO, Suzhou, Jiangsu (CN); BO TANG, Suzhou, Jiangsu (CN)

(21) Appl. No.: 17/795,856

(22) PCT Filed: Nov. 9, 2021
(86) PCT No.: PCT/CN2021/129617

§ 371 Date: Jul. 27, 2022

(30) Foreign Application Priority Data
Oct. 23, 2021 (CN) 202111236629.2

(51) Int. Cl. B01L 3/00

(52) U.S. Cl. CPC B01L 3/523; B01L 2200/0689

(57) ABSTRACT

The present disclosure provides a highly sealed PCR single tube, which relates to the technical field of the PCR single tube. The PCR single tube comprises a sealing tube, wherein the sealing tube is movably placed inside a storage box, four limited posts are fixedly connected on an upper surface of the storage box, a box cover is movably connected at upper ends of the limited posts, and an auxiliary component is fixedly installed inside the box cover. The sealing tube comprises a stud that is movably connected with a screw tube, four movable blocks are movably installed inside a lower side of the screw tube, and a second hose is fixedly connected outside the screw tube. The sealing tube comprises the stud that is movably connected with the screw tube, so that the stud could move up and down through rotation.

### TECHNICAL FIELD

The present disclosure relates to the technical field of a PCR single tube, and specifically relates to a highly sealed PCR single tube.

### BACKGROUND

A PCR single tube is required in the process of closed tube detection of a PCR single tube. For example, a PCR single tube with high stability strength with an application number of CN201810551911.1 relates to a two-dimensional PCR single tube reaction system, method, and kit for closed tube detection of multiple HPV subtypes. The two-dimensional PCR reaction system for closed tube detection of multiple HPV subtypes includes upstream primers of all HPV subtypes to be detected. The upstream primer of each HPV subtype to be detected is provided with a label, and a single nucleotide polymorphic site is formed on the label of each HPV subtype to be detected, so that each HPV subtype to be detected has different melting temperatures. It has the advantages that: five HPV genotypes for closed tube detection of a high-throughput two-dimensional PCR single tube are established for early screening and prevention of condyloma acuminatum; and in accordance with the two-dimensional PCR technology based on a base quenching probe, a fluorescence channel is used to detect multiple genes according to the principle that one target gene corresponds to the melting temperature and the fluorescence detection channel at the same time.

However, in the above technical solution, when the materials to be detected are stored, the sealing effect is general, which may lead to the change in the chemical properties of the materials and the deviation of detection results. Therefore, the PCR single tube with sealing effect and auxiliary device thereof are proposed here.

### SUMMARY

### (I) Technical Problems to be Solved

In view of the insufficiency of the prior art, the present disclosure provides a highly sealed PCR single tube, which solves the problem that when the traditional PCR single tube is used to store the materials to be detected, the sealing effect is general, which may lead to the change in chemical properties of the materials and the deviation of detection results.

### (II) Technical Solution

The present disclosure adopts the following technical solutions to achieve the above purpose: A highly sealed PCR single tube, comprising a sealing tube, wherein the sealing tube is movably placed inside a storage box, four limited posts are fixedly connected on an upper surface of the storage box, a box cover is movably connected at upper ends of the limited posts, and an auxiliary component is fixedly installed inside the box cover; and the sealing tube comprises a stud that is movably connected with a screw tube, four movable blocks are movably installed inside a lower side of the screw tube, a second hose is fixedly connected outside the screw tube, and a connecting belt is fixedly connected outside the screw tube.

Preferably, the auxiliary component comprises a motor, and a first gear is fixedly connected at an output end of the motor.

Preferably, the first gear is respectively in mesh connection with four second gears, and a rotating rod is fixed connected at upper ends of the second gears and movably installed inside the box cover.

Preferably, a first spring is fixedly connected on a lower surface of the box cover, and the second gears are movably connected on one side of the first spring away from the box cover.

Preferably, a cross plate is fixedly connected on a lower side of the second gear, a cross groove is arranged inside an upper side of the screw tube, and the cross plate is movably connected to the cross groove.

Preferably, a limit ring is arranged outside the second hose, and a first hose is movably connected outside the second hose.

Preferably, a second slot is formed inside the first hose, and the limit ring is movably connected inside the second slot.

Preferably, a fixing tube is fixedly connected outside the first hose, a fixing ring outside the fixing tube is connected to one end of the connecting belt, and a liquid storage tube is fixedly connected on a lower side of the first hose.

Preferably, a first slot is formed outside the liquid storage tube, and a plurality of arc-shaped clamping plates are movably installed inside the storage box through a second spring.

Preferably, the liquid storage tube is movably connected inside the four arc-shaped clamping plates, four limit plates are fixedly connected outside the liquid storage tube, and the liquid storage tube is placed in the storage box in a limiting manner through the limiting plates.

Compared with the prior art, the present disclosure has the following beneficial effects:

According to the present disclosure, the sealing tube is movably placed in the storage box to support the sealing tube and prevent it from tipping; and the four limited posts are fixedly connected on the upper surface of the storage box, and the box cover is movably connected at the upper ends of the limited posts, so that users can fetch the sealing tube by vertically moving the box cover. The sealing tube comprises the stud that is movably connected with the screw tube, so that the stud can move up and down through rotation; and the four movable blocks are movably installed inside the lower side of the screw tube, and the second hose is fixedly connected outside the screw tube, so that when the stud moves downwards, the four movable blocks are extruded outwards, and then the second hose could be firmly in a limited connection with the liquid storage tube of the sealing tube, thus enhancing the tightness of the PCR single tube. Different from traditional PCR single tubes, the storage failure of the highly sealed PCR single tube is longer when storing materials, and the internal materials do not lead to detection data errors due to the change in chemical properties caused by poor tightness;

According to the present disclosure, the auxiliary component comprises the motor, and the first gear is fixed connected at the output end of the motor, so that the users can control the rotation of the first gear by controlling the motor; and the first gear is respectively in mesh connection with the four second gears, and the rotating rod is fixedly connected at the upper ends of the second gears and movably installed inside the box cover, so that the first gear can rotate with the second gears, and when rotating, the second gears can move up and down. The first spring is fixedly connected on the lower surface of the box cover, and the second gears are movably connected on one side of the first spring away from the box cover, so that the second gears are subjected to the downward acting force of the first spring when rotating; and the cross plate is fixedly connected to the lower side of the second gear, the cross groove is arranged inside the upper side of the screw tube, and the cross plate is movably connected to the cross groove, so that the second gears can rotate with the stud and move downwards to assist the second hose of the sealing tube in doing a sealing action. The limit ring is arranged outside the second hose, so that the limit ring is forced to expand outwards when the stud descends; and the first hose is movably connected outside the second hose, the second slot is formed inside the first hose, and the limit ring is movably connected inside the second slot, so that the limit ring could be in close connection with the second slot to fill the second slot, thereby greatly improving the tightness of the PCR single tube. The fixing tube is fixedly connected outside the first hose, the fixing ring outside the fixing tube is connected to one end of the connecting belt to support the first hose; and the liquid storage tube is fixedly connected on the lower side of the first hose, the first slot is formed outside the liquid storage tube, and the plurality of arc-shaped clamping plates are movably installed inside the storage box through the second spring, so that the sealing tube could pass through the arc-shaped clamping plate when being placed in the storage box to limit the sealing tube, thus preventing the sealing tube from falling out of the storage box when shaking violently to cause the leakage of internal materials. The liquid storage tube is movably connected inside the four arc-shaped clamping plates, and the four limit plates are fixedly connected outside the liquid storage tube, so that when the sealing tube could be limited and positioned through the limit plate when being placed into a placing groove stored inside the storage box, thereby facilitating the auxiliary component to seal the sealing tube; and a plurality of sealing tubes could be sealed at one time, thereby accelerating the work efficiency.

### BRIEF DESCRIPTION OF DRAWINGS

FIG. 1 is a schematic diagram for an overall structure of a highly sealed PCR single tube of the present disclosure;

FIG. 2 is a schematic diagram for an internal structure of a highly sealed PCR single tube of the present disclosure;

FIG. 3 is a schematic diagram for a partial structure of an auxiliary component of a highly sealed PCR single tube of the present disclosure;

FIG. 4 is a structural diagram for a sealing tube of a highly sealed PCR single tube of the present disclosure;

FIG. 5 is a structural diagram for a liquid storage tube of a highly sealed PCR single tube of the present disclosure;

FIG. 6 is a structural diagram for a stud of a highly sealed PCR single tube of the present disclosure.

In the Figure: 1. storage box; 2. limited post; 3. box cover; 4. sealing tube; 401. liquid storage tube; 402. limit plate; 403. first slot; 404. second slot; 405. connecting belt; 406. stud; 407. first hose; 408. fixing tube; 409. cross groove; 410. screw tube; 411. movable block; 412. second hose; 413. limit ring; 414. first spring; 5. auxiliary component; 501, motor; 502. first gear; 503. second gear; 504. rotating rod; 505. cross plate; 506. second spring; 6. arc-shaped clamping plate.

### DETAILED DESCRIPTION

Technical solutions in embodiments of the present disclosure are clearly and completely described with reference to the embodiments of the present disclosure. Apparently, the embodiments described below are merely part, not all, of the embodiments of the present disclosure. Based on the embodiments described herein, all other embodiments obtained by those of ordinary skill in the art without creative work are within the scope of the present disclosure.

### EMBODIMENT

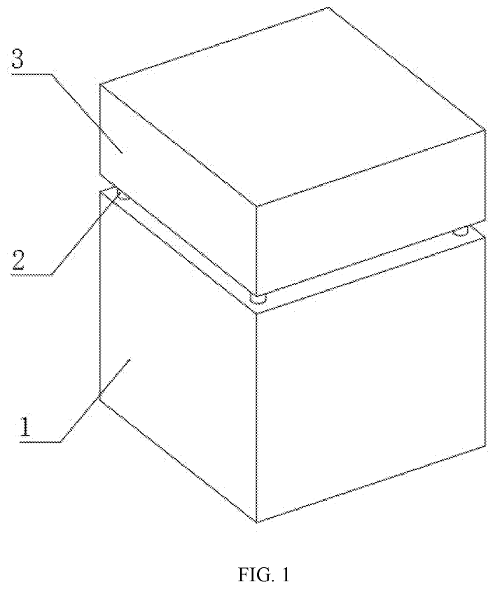

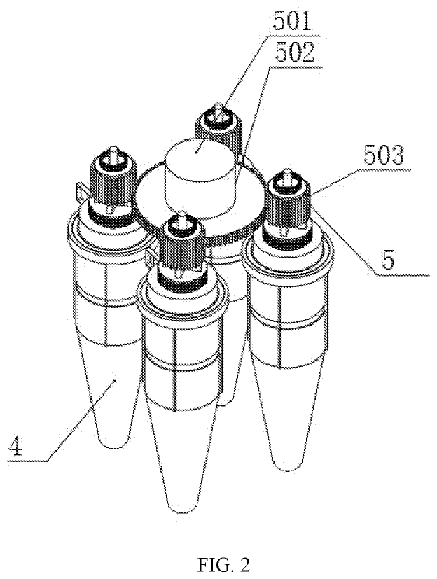

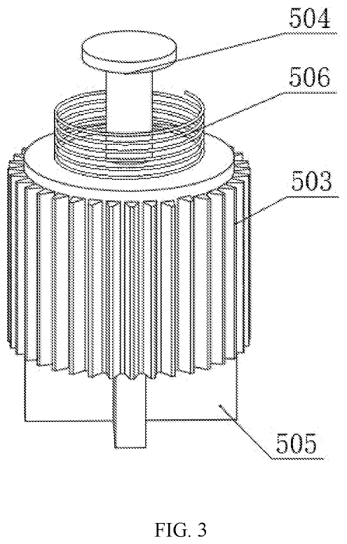

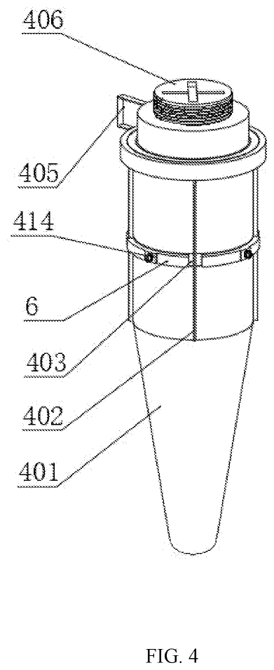

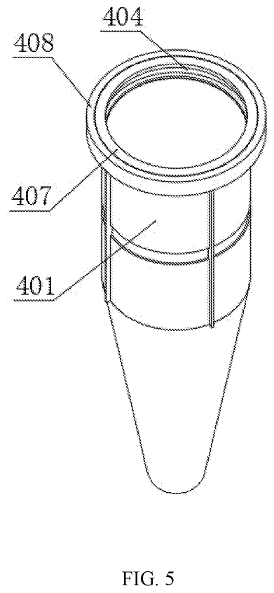

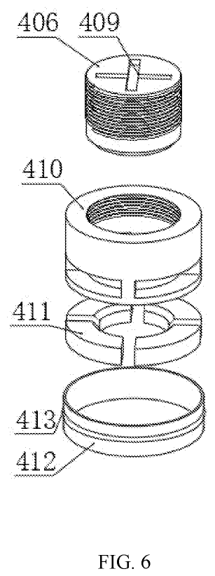

As shown in FIGS. 1-6, a highly sealed PCR single tube, comprising a sealing tube 4, wherein the sealing tube 4 is movably placed inside a storage box 1, four limited posts 2 are fixedly connected on an upper surface of the storage box 1, a box cover 3 is movably connected at upper ends of the limited posts 2, and an auxiliary component 5 is fixedly installed inside the box cover 3; and the sealing tube 4 comprises a stud 406 that is movably connected with a screw tube 410, four movable blocks 411 are movably installed inside the lower side of the screw tube 410, a second hose 412 is fixedly connected outside the screw tube 410, and a connecting belt 405 is fixedly connected outside the screw tube 410.

According to the technical solution, the sealing tube 4 is movably placed in the storage box 1 to support the sealing tube 4 and prevent it from tipping; and the four limited posts 2 are fixedly connected on the upper surface of the storage box 1, and the box cover 3 is movably connected at the upper ends of the limited posts 2, so that users can fetch the sealing tube 4 by vertically moving the box cover 3. The sealing tube 4 comprises the stud 406 that is movably connected with the screw tube 410, so that the stud 406 can move up and down through rotation; and the four movable blocks 411 are movably installed inside the lower side of the screw tube 410, and the second hose 412 is fixedly connected outside the screw tube 410, so that when the stud 406 moves downwards, the four movable blocks 411 are extruded outwards, and then the second hose 412 could be firmly in a limited connection with the liquid storage tube 401 of the sealing tube 4, thus enhancing the tightness of the PCR single tube. Different from traditional PCR single tubes, the storage failure of the highly sealed PCR single tube is longer when storing materials, and the internal materials do not lead to detection data errors due to the change in chemical properties caused by poor tightness.

In this embodiment, the auxiliary component 5 comprises a motor 501, a first gear 502 is fixedly connected at an output end of the motor 501, the first gear 502 is respectively in mesh connection with four second gears 503, a rotating rod 504 is fixedly connected at upper ends of the second gears 503, and the rotating rod 504 is movably installed inside the box cover 3. The auxiliary component 5 comprises the motor 501, and the first gear 502 is fixedly connected at the output end of the motor 501, so that the users can control the rotation of the first gear 502 by controlling the motor 501; and the first gear 502 is respectively in mesh connection with the four second gears 503, the rotating rod 504 is fixedly connected at the upper ends of the second gears 503, and the rotating rod 504 is movably installed inside the box cover 3, so that the first gear 502 can rotate with the second gears 503, and when rotating, the second gears 503 can move up and down.

In this embodiment, a first spring 414 is fixedly connected on the lower surface of the box cover 3, the second gears 503 are movably connected on one side of the first spring 414 away from the box cover 3, a cross plate 505 is fixedly connected to the lower side of the second gear 503, and a cross groove 409 is arranged inside the upper side of the screw tube 410, and the cross plate 505 is movably connected to the cross groove 409. The first spring 414 is fixedly connected on the lower surface of the box cover 3, and the second gears 503 are movably connected on one side of the first spring 414 away from the box cover 3, so that the second gears 503 are subjected to the downward acting force of the first spring 414 when rotating; and the cross plate 505 is fixedly connected to the lower side of the second gear 503, the cross groove 409 is arranged inside the upper side of the screw tube 410, and the cross plate 505 is movably connected to the cross groove 409, so that the second gears 503 can rotate with the stud 406 and move downwards to assist the second hose 412 of the sealing tube 4 in doing a sealing action.

In this embodiment, a limit ring 413 is arranged outside the second hose 412, a first hose 407 is movably connected outside the second hose 412, a second slot 404 is formed inside the first hose 407, and the limit ring 413 is movably connected inside the second slot 404. The limit ring 413 is arranged outside the second hose 412, so that the limit ring 413 is forced to expand outwards when the stud 406 descends; and the first hose 407 is movably connected outside the second hose 412, the second slot 404 is formed inside the first hose 407, and the limit ring 413 is movably connected inside the second slot 404, so that the limit ring 413 could be in close connection with the second slot 404 to fill the second slot 404, thereby greatly improving the tightness of the PCR single tube.

In this embodiment, a fixing tube 408 is fixedly connected outside the first hose 407, a fixing ring outside the fixing tube 408 is connected to one end of the connecting belt 405, a liquid storage tube 401 is fixedly connected on a lower side of the first hose 407, a first slot 403 is formed outside the liquid storage tube 401, and a plurality of arc-shaped clamping plates 6 are movably installed inside the storage box 1 through a second spring 506. The fixing tube 408 is fixedly connected outside the first hose 407, the fixing ring outside the fixing tube 408 is connected to one end of the connecting belt 405 to support the first hose 407; and the liquid storage tube 401 is fixedly connected on the lower side of the first hose 407, the first slot 403 is formed outside the liquid storage tube 401, and the plurality of arc-shaped clamping plates 6 are movably installed inside the storage box 1 through the second spring 506, so that the sealing tube 4 could pass through the arc-shaped clamping plate 6 when being placed in the storage box 1 to limit the sealing tube 4, thus preventing the sealing tube 4 from falling out of the storage box 1 when shaking violently to cause the leakage of internal materials.

In this embodiment, the liquid storage tube 401 is movably connected inside the four arc-shaped clamping plates 6, four limit plates 402 are fixedly connected outside the liquid storage tube 401, and the liquid storage tube 401 is placed in the storage box 1 in a limiting manner through the limiting plates 402. The liquid storage tube 401 is movably connected inside the four arc-shaped clamping plates 6, and the four limit plates 402 are fixedly connected outside the liquid storage tube 401, so that when the sealing tube 4 could be limited and positioned through the limit plate 402 when being placed into a placing groove stored inside the storage box 1, thereby facilitating the auxiliary component 5 to seal the sealing tube 4; and a plurality of sealing tubes 4 could be sealed at one time, thereby accelerating the work efficiency.

The highly sealed PCR single tube has the working principles that:

When in use, firstly, the sealing tube 4 is movably placed in the storage box 1 to support the sealing tube 4 and prevent it from tipping; and four limited posts 2 are fixedly connected on the upper surface of the storage box 1, and the box cover 3 is movably connected at the upper ends of the limited posts 2, so that users can fetch the sealing tube 4 by vertically moving the box cover 3. The sealing tube 4 comprises the stud 406 that is movably connected with the screw tube 410, so that the stud 406 can move up and down through rotation; and four movable blocks 411 are movably installed inside the lower side of the screw tube 410, and the second hose 412 is fixedly connected outside the screw tube 410, so that when the stud 406 moves downwards, the four movable blocks 411 are extruded outwards, and then the second hose 412 could be firmly in a limited connection with the liquid storage tube 401 of the sealing tube 4, thus enhancing the tightness of the PCR single tube. Different from traditional PCR single tubes, the storage failure of the highly sealed PCR single tube is longer when storing materials, and the internal materials do not lead to detection data errors due to the change in chemical properties caused by poor tightness. The auxiliary component 5 comprises the motor 501, and the first gear 502 is fixedly connected at the output end of the motor 501, so that the users can control the rotation of the first gear 502 by controlling the motor 501; and the first gear 502 is respectively in mesh connection with the four second gears 503, the rotating rod 504 is fixed connected at the upper ends of the second gears 503, and the rotating rod 504 is movably installed inside the box cover 3, so that the first gear 502 can rotate with the second gears 503, and when rotating, the second gears 503 can move up and down. The first spring 414 is fixedly connected on the lower surface of the box cover 3, and the second gears 503 are movably connected on one side of the first spring 414 away from the box cover 3, so that the second gears 503 are subjected to the downward acting force of the first spring 414 when rotating; and the cross plate 505 is fixedly connected to the lower side of the second gear 503, the cross groove 409 is arranged inside the upper side of the screw tube 410, and the cross plate 505 is movably connected to the cross groove 409, so that the second gears 503 can rotate with the stud 406 and move downwards to assist the second hose 412 of the sealing tube 4 in doing a sealing action. The limit ring 413 is arranged outside the second hose 412, so that the limit ring 413 is forced to expand outwards when the stud 406 descends; and the first hose 407 is movably connected outside the second hose 412, the second slot 404 is formed inside the first hose 407, and the limit ring 413 is movably connected inside the second slot 404, so that the limit ring 413 could be in close connection with the second slot 404 to fill the second slot 404, thereby greatly improving the tightness of the PCR single tube. The fixing tube 408 is fixedly connected outside the first hose 407, the fixing ring outside the fixing tube 408 is connected to one end of the connecting belt 405 to support the first hose 407; and the liquid storage tube 401 is fixedly connected on the lower side of the first hose 407, the first slot 403 is formed outside the liquid storage tube 401, and the plurality of arc-shaped clamping plates 6 are movably installed inside the storage box 1 through the second spring 506, so that the sealing tube 4 could pass through the arc-shaped clamping plate 6 when being placed in the storage box 1 to limit the sealing tube 4, thus preventing the sealing tube 4 from falling out of the storage box 1 when shaking violently to cause the leakage of internal materials. The liquid storage tube 401 is movably connected inside the four arc-shaped clamping plates 6, and the four limit plates 402 are fixedly connected outside the liquid storage tube 401, so that when the sealing tube 4 could be limited and positioned through the limit plate 402 when being placed into a placing groove stored inside the storage box 1, thereby facilitating the auxiliary component 5 to seal the sealing tube 4; and a plurality of sealing tubes 4 could be sealed at one time, thereby accelerating the work efficiency.

It should be noted that the specific model of the motor 501 is Y112M-4 4KWB3.

The basic principle, main features, and advantages of the present disclosure are shown and described above. Those skilled in the art should know that the present disclosure is not limited by the above embodiments. The above embodiments and description only describe the principles of the present disclosure. Without departing from the spirit and scope of the present disclosure, various changes and improvements will also be made to the present disclosure, and fall within the requested scope of the present disclosure. The scope of protection requested by the present disclosure is defined by the attached claim and its equivalents.

1. A highly sealed PCR single tube, comprising a sealing tube (4), wherein the sealing tube (4) is movably placed inside a storage box (1), four limited posts (2) are fixedly connected on an upper surface of the storage box (1), a box cover (3) is movably connected at upper ends of the limited posts (2), and an auxiliary component (5) is fixedly installed inside the box cover (3); and the sealing tube (4) comprises a stud (406) that is movably connected with a screw tube (410), four movable blocks (411) are movably installed inside a lower side of the screw tube (410), a second hose (412) is fixedly connected outside the screw tube (410), and a connecting belt (405) is fixedly connected outside the screw tube (410).

2. The highly sealed PCR single tube according to claim 1, wherein the auxiliary component (5) comprises a motor (501), and a first gear (502) is fixedly connected at an output end of the motor (501).

3. The highly sealed PCR single tube according to claim 2, wherein the first gear (502) is respectively in mesh connection with four second gears (503), and a rotating rod (504) is fixed connected at upper ends of the second gears (503) and movably installed inside the box cover (3).

4. The highly sealed PCR single tube according to claim 3, wherein a first spring (414) is fixedly connected on a lower surface of the box cover (3), and the second gears (503) are movably connected on one side of the first spring (414) away from the box cover (3).

5. The highly sealed PCR single tube according to claim 4, wherein a cross plate (505) is fixedly connected on a lower side of the second gear (503), a cross groove (409) is arranged inside an upper side of the screw tube (410), and the cross plate (505) is movably connected to the cross groove (409).

6. The highly sealed PCR single tube according to claim 1, wherein a limit ring (413) is arranged outside the second hose (412), and a first hose (407) is movably connected outside the second hose (412).

7. The highly sealed PCR single tube according to claim 6, wherein a second slot (404) is formed inside the first hose (407), and the limit ring (413) is movably connected inside the second slot (404).

8. The highly sealed PCR single tube according to claim 7, wherein a fixing tube (408) is fixedly connected outside the first hose (407), a fixing ring outside the fixing tube (408) is connected to one end of the connecting belt (405), and a liquid storage tube (401) is fixedly connected on a lower side of the first hose (407).

9. The highly sealed PCR single tube according to claim 8, wherein a first slot (403) is formed outside the liquid storage tube (401), and a plurality of arc-shaped clamping plates (6) are movably installed inside the storage box (1) through a second spring (506).

10. The highly sealed PCR single tube according to claim 9, wherein the liquid storage tube (401) is movably connected inside the four arc-shaped clamping plates (6), four limit plates (402) are fixedly connected outside the liquid storage tube (401), and the liquid storage tube (401) is placed in the storage box (1) in a limiting manner through the limit plate (402).
